(19) United States
(12) Patent Application Publication
(10) Pub. No.: US 2026/0260709 A1
(43) Pub. Date: Sep. 3, 2026

(54) Processes and Systems for Predicting, Prioritizing, and Designing Broad-Spectrum Antibodies Using Machine Learning

(71) Applicant: The Texas A&M University System, College Station, TX (US)

(72) Inventors: Yang SHEN, College Station, TX (US); Yuanfei SUN, St. George, UT (US); Wuwei TAN, Beijing (CN)

(21) Appl. No.: 19/555,316

(22) Filed: Mar. 3, 2026

Related U.S. Application Data
(60) Provisional application No. 63/766,218, filed on Mar. 3, 2025.

(51) Int. Cl. G16B 40/30; G06N 3/088

(52) U.S. Cl. CPC G16B 40/30; G06N 3/088

(57) ABSTRACT

A computer-implemented method of building and training an antibody language model (AbLM) to provide antibody screening or design for virus neutralization is provided. The method includes receiving unlabeled data comprising single-chain protein sequences; training a protein language model (pLM) using the unlabeled data; initializing an AbLM using the trained pLM; applying complementary-determining region (CDR) masking to a plurality of variable heavy (VH) and variable light (VL) chain sequences; training the AbLM using the plurality of CDR-masked VH and VL chain sequences in paired form; and applying the trained AbLM for at least one of antibody screening or antibody design.

### CROSS-REFERENCE TO RELATED APPLICATIONS

The present application claims priority to and the benefit of U.S. Provisional Patent Application No. 63/766,218 filed Mar. 3, 2025, and entitled “Processes and Systems for Predicting, Prioritizing, and Designing Broad-Spectrum Antibodies using Machine Learning,” which is hereby incorporated herein by reference in its entirety as if fully set forth below and for all applicable purposes.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

An antibody is a protein produced by the body's immune system when it detects harmful substances, called antigens. Examples of antigens may include microorganisms, such as bacteria, fungi, parasites, and viruses. Antibodies are widely used in therapeutic and diagnostic applications because of their ability to recognize target antigens, allowing selective modulation or neutralization of disease-associated molecules while limiting unintended interactions. Additionally, antibodies can be engineered to enhance potency, extend half-life, or recruit immune effector functions, making them versatile agents for a broad range of clinical applications.

### SUMMARY

In an embodiment, a computer-implemented method of building and training an antibody language model (AbLM) to provide antibody screening or design for virus neutralization is provided. The method includes receiving, by an application stored in a non-transitory memory of a computer system and executed by a processor of the computer system, unlabeled data comprising single-chain protein sequences; training, by the application, a protein language model (pLM) using the unlabeled data; initializing, by the application, an AbLM using the trained pLM; applying, by the application, complementary-determining region (CDR) masking to a plurality of variable heavy (VH) and variable light (VL) chain sequences; training, by the application, the AbLM using the plurality of CDR-masked VH and VL chain sequences in paired form; and applying, by the application, the trained AbLM for at least one of antibody screening or antibody design.

In another embodiment, a computer-implemented method of performing machine learning-based antibody selection for a target virus using an antibody language model (AbLM) is provided. The method includes receiving, by an application stored in a non-transitory memory of a computer system and executed by a processor of the computer system, a plurality of antibody sequences; encoding, by the application, the plurality of antibody sequences into respective feature representations using the AbLM; for each of the plurality of antibody sequences, predicting, by the application, based on the encoding, respective activity against a target virus using an activity prediction model; and selecting at least one antibody sequence of the plurality of antibody sequences based on respective predicted activity against the target virus.

In yet another embodiment, a computer-implemented method of using an antibody language model (AbLM) for antibody design is provided. The method includes receiving, by an application stored in a non-transitory memory of a computer system and executed by a processor of the computer system, an antibody sequence; masking, by the application, a portion of the antibody sequence; initiating, by the application, an AbLM to process the antibody sequence; receiving, by the application, from the AbLM, one or more outputs comprising at least one of encoded features of the antibody sequence or a probability distribution of amino acid at each sequence position of the antibody sequence; and generating, by the application, one or more antibodies for a target virus based on the one or more outputs of the AbLM.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, where like reference numerals represent like parts.

FIG. 1 is a block diagram illustrating a network system for implementing data-driven, machine learning (ML)-based screening, design, and/or prioritization of broad-spectrum antibodies according to an embodiment of the present disclosure.

FIG. 2 illustrates a framework for analyzing broad-spectrum antibodies using physics-driven and data-driven, ML processes according to an embodiment of the present disclosure.

FIG. 3 illustrates an example method of training an antibody language model (AbLM) to enable screening, design, and/or prioritization of broad-spectrum antibodies according to an embodiment of the present disclosure

FIG. 4 illustrates an example method of using an AbLM for screening, design, and/or prioritization of broad-spectrum antibodies according to an embodiment of the present disclosure

FIG. 5 illustrates an example method of training a prediction model to predict antibody activity for a target virus according to an embodiment of the present disclosure.

FIG. 6 illustrates an example method of training a hybrid AbLM-prediction model to predict antibody activity for a target virus according to an embodiment of the present disclosure.

FIG. 7 illustrates an example method of using an AbLM to design broad-spectrum antibodies for a target virus according to an embodiment of the present disclosure.

FIG. 8 is a block diagram illustrating an AbLM with sequence concatenation according to an embodiment of the present disclosure.

FIG. 9 is a flow chart of a method according to an embodiment of the disclosure.

FIG. 10 is a flow chart of another method according to an embodiment of the disclosure.

FIG. 11 is a flow chart of yet another method according to an embodiment of the disclosure.

FIG. 12 is a block diagram of a computer system according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

As used herein, the term “labeled data” may refer to data including protein and/or antibody sequences and corresponding labels or annotations that characterize profiles and/or activity of the respective sequence (e.g., against a certain antigen or virus).

Therapeutic antibodies are widely used in modern medicine as agents for treating infectious pathogens, cancer, and many other diseases. However, experimental screening for highly efficacious targeting antibodies can be labor-intensive, time-consuming, and costly, which is exacerbated by evolving antigen targets under selective pressure such as fast-mutating viral variants. Recent advances in computational biology and machine learning (ML) have enabled development of protein and antibody modeling and design approaches that leverage large-scale protein sequence data (e.g., public databases). However, there are technical challenges in developing ML models for antibody analysis and/or for anticipating the effects of potential mutations. One such challenge is the lack of and/or limited amount of publicly accessible antibody data, and in particular, activity data (i.e., experimental measurements for each antibody). The limitation in publicly accessible activity data becomes especially problematic in early response scenarios, such as when a new disease or a novel viral strain emerges. As a result, labeled data (including antibodies with characterized functional profiles or activities) are often limited during periods of heightened urgency in antibody screening and antibody design.

The present disclosure is directed to an antibody language model (AbLM) and particular uses of an AbLM. To overcome the aforementioned small data challenge, the AbLM is built in part using unlabeled public data (e.g., protein sequences or structures and antibody sequences or structures). According to an embodiment of the present disclosure, the AbLM is built in part using one or more protein language models (pLMs) trained on unlabeled protein sequence data. Stated differently, the pLM may be used to warm start the AbLM. For instance, an application (e.g., software executing on a computer system) may pre-train a pLM using unlabeled data comprising protein sequences (e.g., single-chain protein sequences). In an embodiment, the pLM may be a transformer encoder. The application may initialize an AbLM using the pre-trained pLM and fine-tune the AbLM using unlabeled antibody data comprising paired variable heavy (VH) and variable light (VL) chain sequences. Since there is a greater amount of unlabeled protein data (e.g., including millions of single-chain protein sequences) available than unlabeled antibody data (e.g., including thousands of paired VH and VL chain sequences), pre-training a pLM using the unlabeled protein data and initializing the AbLM from the pre-trained pLM enables the AbLM to be fine-tuned using a much smaller set of unlabeled antibody data, thereby addressing part of the small data challenge discussed above.

The fine-tuning of the AbLM may be based on self-supervised learning. For instance, as part of fine-tuning the AbLM, the application may apply complementary-determining region (CDR) masking to a plurality of VH and VL chain sequences and train the AbLM using the plurality of CDR-masked VH and VL chain sequences in paired form. CDRs are immunoglobulin hypervariable domains that determine specific antibody binding pattern towards antigens. In an embodiment, as part of applying CDR masking, the application may select one CDR region (e.g., randomly) and mask all amino acids within the one CDR region. Generally, the application may use any type of inductive bias masking strategy to fine-tune and/or train the AbLM. Example masking strategies may include, but are not limited to, a “vanilla” strategy, a “CDR-vanilla” strategy, a “CDR-margin” strategy, and a “CDR-pair” strategy. The “vanilla” strategy may apply a masking strategy that is used in bidirectional encoder representations from transformers (BERT) over the whole VH or VL chain sequences. For example, in one embodiment, 15% of amino acids may be masked, among those, 80% of amino acids may be replaced with <MASK> token, 10% of amino acids may be replaced by other random amino acids, and 10% of amino acids may remain unchanged. The “CDR-vanilla” strategy may apply the aforementioned BERT masking strategy in six CDR regions. The “CDR-margin” strategy may randomly select one CDR region (out of six) and mask all amino acids within the selected CDR region. The “CDR-pair” strategy may mask all amino acids in one pair of VH-VL CDR regions randomly selected among six CDR regions. In various embodiments, a number of potential additional masking techniques or combinations of techniques may be used. For example, 5% of amino acids outside CDR regions may be masked for any of the CDR biased strategies to enable density estimation of fairly constant framework regions.

As part of training the AbLM, the application may input a pair of the CDR-masked VH and VL chain sequences separately into respective ones of weight-tied pLMs to generate corresponding VH embedding and VL embedding. The weight-tied pLMs may refer to two identical pLMs with identical parameters (e.g., weights and/or biases) initialized based on the pre-trained pLM. The VH embedding may correspond to a feature vector representative of the VH chain sequence of the VH-VL sequence pair in a latent space. Similarly, the VL embedding may correspond to a feature vector representative of the VL chain sequence of the sequence pair in the latent space. The application may further apply a cross-attention fusion model to the VH embedding and the VL embedding. In an example, the cross-attention fusion model may include bidirectional transformer encoders (e.g., with 12 layers and 12 heads per layer). The cross-attention fusion model may allow information to flow between the two chains (e.g., the VH embedding and the VL embedding). Stated differently, the cross-attention fusion model may capture pairwise dependencies and structural relationships between the two VH and VL chain sequences. The cross-attention fusion model may update the VH embedding based on the VL embedding and/or update the VL embedding based on the VH embedding. The cross-attention fusion model may output an antibody embedding by concatenating the updated VH embedding and the updated VL embedding. The application may further apply a projector or masked language model (MLM) head to the antibody embedding to generate an output comprising amino acid probabilities (e.g., a probability distribution over amino acids) at each sequence position of the antibody sequence. Stated differently, as part of training the AbLM, some portions of each of the VH sequence and VL sequence in a VH-VL sequence pair may be blanked-out and fed to the AbLM (including the transformer encoder or pLM and the cross-attention fusion model) and the AbLM may predict what the blanks (e.g., the amino acids) should be based on the non-blanked portions.

As part of training the AbLM, the application may further adapt parameters of the weight-tied pLMs based on an error measurement between the output of the AbLM and the input pair of VH and VL chain sequences (without the CDR masking). The error measurement may be computed based on a CDR loss function (e.g., measuring loss in the masked region(s) or CDR(s)). In an example, the loss function may compute a cross-entropy loss over masked positions, comparing the predicted probability distribution over tokens with a true token (e.g., representing the actual amino acid in the input sequence) at each masked position. The CDR masking and the AbLM processing may be repeated over multiple iterations for a pair of VH and VL chain sequences until the error measurement satisfies certain criteria (e.g., below a certain threshold) and may be further repeated for each pair of VH and VL chain sequences in the plurality of VH and VL chain sequences. In the initial iteration of the training, each of the weight-tied pLMs may correspond to the pre-trained pLM.

In another embodiment, the AbLM may include a single pLM (e.g., initialized using the pre-trained pLM) and a cross-attention fusion model. In such an embodiment, the application may process a pair of the CDR-masked VH chain sequence and the VL chain sequence separately using the pLM in a sequential manner to generate corresponding VH embedding and VL embedding and apply the cross-attention fusion model as discussed above. In some instances, the weight-tied pLM or the single pLM with sequential processing may be selected to trade-off between memory usage and computational speed. In yet another embodiment, the AbLM may also include a single pLM (e.g., initialized using the pre-trained pLM) but without a cross-attention fusion model. In such an embodiment, the application may concatenate the VH chain sequence and the VL sequence in a VH-VL sequence pair with a special token, <SEP>, which may enable the VH and VL chain sequences to be processed by the single pLM as a single sequence input. A special token is a token that is not present in protein sequences and does not correspond to a specific amino-acid type. Inserting a special token between the two VH and VL sequences can provide an indication of boundary between the two sequences. In an example, one or more special tokens may be used to concatenate the VH chain sequence and the VL chain sequence, where the one or more tokens may be padding tokens, <PAD>, to change a variable-length sequence into a fixed-length sequence (with predetermined length). In an example, the concatenation may use two special amino acids (U and O), three ambiguity letters (X, B, and Z), and five special tokens including <PAD> and <SEP>.

In some embodiments, the AbLM may be used in combination with a predictor (or decoder). The pre-trained AbLM may be fixed (e.g., with fixed parameters) as a texturizer for any antibody sequence input and the predictor is trained on few labeled data such as antibody activity profiles against WT and variants, whether experimentally measured or computationally predicted (including predictions from the predictor itself trained on the previous round). The predictor may be a nonparametric Gaussian regressor such as Kriging. Kriging uses few parameters, which may be beneficial and assist in addressing the small data challenge discussed above. There are also efficiencies gained by using a simpler ML such as Kriging (e.g., processing efficiencies, memory efficiencies, etc.). However, other ML models may be used for the predictor as well. In some instances, the predictor may also be referred to as an activity prediction model. Training the predictor separately after the AbLM is trained can enable the predictor to be trained using a substantially smaller set of labeled data or activity data (e.g., include tens of antibody sequences with corresponding activity information), which may be beneficial and may assist in addressing the small data challenge discussed above.

In other embodiments, the AbLM and the predictor may be jointly trained. In such embodiments, the predictor may include a few layers of neural networks (i.e., prediction heads) in serial connection to the AbLM. The AbLM may be a fine-tunable encoder and the encoder and predictor may be trained together using labeled data discussed above to create a hybrid antibody model and predictor. Prediction heads may use much less parameters compared to the encoder, whereas the encoder may be pre-trained using massive unlabeled data and thus warm-started during fine-tuning using few labeled data, which may be beneficial and may assist in addressing the small data challenge discussed above.

After the AbLM and the predictor or their hybrid is trained, the AbLM model and/or the predictor may be applied in different ways. The AbLM alone or in combination with the predictor may be used to predict known antibody sequences and design for new antibody sequences. For instance, the AbLM may receive as input a complete antibody sequence (e.g., paired VH-VL chain sequences). The AbLM may provide different kinds of output such as (1) embedding representing the antibody sequence and/or (2) amino acid probabilities at each sequence position. The AbLM can produce these outputs even with limited or no experimental data initially. The outputs of the AbLM may be used for antibody engineering maturation. The AbLM may be used with the predictor to predict activity for any known antibody. For instance, if there is a new virus with a new set of antibodies, the antibodies can be parsed and translated to work with the AbLM, and the AbLM and the predictor may predict the activity expected for each input antibody. The AbLM and the predictor may be used to prioritize known antibodies. The AbLM may be used to design antibodies either from known antibodies (i.e., antibody maturation) or starting from scratch (i.e., de novo design). Feedback about activity for antibody designs may be received from experiments and/or from the AbLM's own prediction (reinforcement learning). The AbLM may be iteratively retrained based on this feedback.

In a first non-limiting example, given a set of antibodies, the AbLM may predict the activity of each antibody against a target virus across multiple strains, including WT, known variants, and anticipated variants. An antibody of the set of antibodies may be selected and used to treat the target virus, and a broad-spectrum antibody may be selected based on robust activities against various strains. Stated differently, the trained AbLM and the trained predictor may be used for antibody screening and/or prioritization.

For instance, for antibody screening, the application may apply the AbLM to an antibody sequence and apply the predictor to the AbLM's output (e.g., an antibody embedding representing the antibody sequence) to predict at least one activity for the antibody sequence. For antibody prioritization, the application may apply the trained AbLM and the predictor to a set of antibody sequences (e.g., known antibodies). The predictor may be trained to predict activities for a target virus (e.g., WT and its variants) and may output an indicator of susceptibility or neutralization efficacy (e.g., a score) of an antibody against the target virus. After applying the trained AbLM and the predictor to each of the plurality of antibody sequences, the application may receive a number of scores for each of the antibody sequences. The application may prioritize and select, based on the scores, at least one antibody sequence from the plurality of antibody sequences that has a clinical potential for improvement. In some embodiments, the application may further adapt one or more parameters of the AbLM based on results of experimentally testing the selected at least one antibody sequence against the target virus. In some embodiments, the selected at least one antibody sequence may be used to treat the target virus. In some embodiments, the selected at least one antibody sequence may be present in a composition further comprising a pharmaceutically acceptable carrier or excipient.

In a second non-limiting example, given a virus, the output(s) of the AbLM may be used to design antibodies (existing or new). For instance, experimentally generated sequences may be input into the AbLM for the AbLM to redesign the sequences and predict their activities. Additionally or alternatively, the output(s) or predictions of the AbLM may be experimentally tested. These two processes may be used iteratively in cycle. In an example, the number of iterations may be predetermined. In an example, an adaptive stopping rule may be applied to terminate the iterations, for example, when there is no significant improvement in new designs or when there no significant changes in the designed sequences. One of the designed antibodies may be selected and used to treat the given virus.

For instance, for antibody design, the application may provide an antibody sequence to the AbLM after masking a portion of the antibody sequence. The masked portion may correspond to a CDR of the antibody sequence. The application may design one or more antibodies for a given virus based on one or more outputs (e.g., the probability distribution of amino acids at each sequence position) of the AbLM. For instance, designing the one or more antibodies may include sampling one or more probability distributions of amino acids at one or more sequence positions (e.g., within the masked portion) of the antibody sequence output by the AbLM. In an example, the sampling may include identifying one or more design (or mutation) positions based on one or more criteria, such as the top-m positions exhibiting the highest amino-acid probability entropy and the number m being fixed or m~Poisson(μ)+1 mutations (subject to a trust radius), where μ is a sequence proposal mutation rate. In an example, the sampling may include selecting an amino acid at each of the one or more sequence positions based on one or more criteria, such as top-K sampling (the most-probable K amino acids are sampled at each design position) where K is a hyperparameter. In an embodiment, the antibody sequence may include an experimentally generated antibody sequence. In some instances, an antibody designed using the AbLM may be fed back to the AbLM as an input to generate another antibody. In an embodiment, the application may retrain the AbLM (e.g., adapting one or more parameters of the AbLM) based on at least one of experimentally testing the one or more designed antibodies against the given virus or evaluating the one or more designed antibodies for virus neutralization using the AbLM and predictor as discussed above. In an embodiment, the application may design multiple antibody sequences using the AbLM and may apply AbLM likelihood-ratio filtering and perform surrogate-model acceptance to select an antibody sequence from the multiple designed antibody sequences. In some embodiments, at least one of the one or more designed antibodies may be present in a composition further comprising a pharmaceutically acceptable carrier or excipient. In some embodiments, at least one of the one or more designed antibodies may be used to treat the given virus. In some embodiments, at least one of the one or more designed antibodies may be present in a composition further comprising a pharmaceutically acceptable carrier or excipient.

In some embodiments, an antibody sequence predicted (or generated) by the AbLM as disclosed herein may advantageously be employed to target a virus or viral pathogen, for example, a virus or viral pathogen employed in the AbLM to generate the antibody. The antibody or a portion thereof (e.g., a predicted portion having activity with respect to the virus) may be included in a composition having a suitable form for delivery to a subject such as a human or other mammal. For example, in various embodiments, a composition may comprise any isolated polypeptide, antibody or antigen-binding fragment thereof as disclosed, any multivalent antibody consistent with this disclosure, or any combination thereof. The composition may further comprise a pharmaceutically acceptable carrier or excipient. The composition may take any suitable form for delivery to the subject, examples of which may include, a gel, an ointment, a liquid, a suspension, an aerosol, a tablet, a pill, a powder, or a nasal spray. In some embodiments, the compositions may be formulated for pulmonary, intranasal, or parenteral administration. The compositions may be formulated for single dosage or multi-dose administration.

In some embodiments, an antibody sequence predicted (or generated) by the AbLM as disclosed herein may advantageously be employed in a method of treating a viral infection in a subject. Additionally or alternatively, in some embodiments, an antibody having a structure (e.g., sequence) generated via the AbLM as disclosed herein may advantageously be employed in a method of treating, lessening, or inhibiting one or more symptoms associated with a viral infection in a subject. Additionally or alternatively, in some embodiments, an antibody having a structure (e.g., sequence) generated via the AbLM as disclosed herein may advantageously be employed in a method of preventing a viral infection in a subject. Generally, the methods disclosed herein may include administering to the subject a therapeutically effective amount of a composition as disclosed herein.

In various embodiments, administration to the subject may be via any suitable route, including but not limited to, topically, parenterally, locally, or systemically, such as for example intranasally, intramuscularly, intradermally, intraperitoneally, intravenously, subcutaneously, orally, or by pulmonary administration. In some embodiments, a pharmaceutical composition provided herein is administered by a nebulizer or an inhaler.

The pharmaceutical compositions provided herein can be administered to any suitable subject, such as a mammal, for example, a human. In various embodiments, the subject may be characterized as having a viral infection, experiencing one or more symptoms associated with a viral infection, and/or at risk for a viral infection. The viral infection may be any viral infection suitable treated via the disclosed antibodies, examples of which may include infections or disease states associated any virus, examples of which include but are not limited to African Swine Fever Viruses, Arbovirus, Adenoviridae, Arenaviridae, Arterivirus, Astroviridae, Baculoviridae, Bimaviridae, Birnaviridae, Bunyaviridae, Caliciviridae, Caulimoviridae, Circoviridae, Coronaviridae, Cystoviridae, Dengue, EBV, HIV, Deltaviridae, Filviridae, Filoviridae, Flaviviridae, Hepadnaviridae (Hepatitis), Herpesviridae (such as, Cytomegalovirus, Herpes Simplex, Herpes Zoster), Iridoviridae, Mononegavirus (e.g., Paramyxoviridae, Morbillivirus, Rhabdoviridae), Myoviridae, Orthomyxoviridae (e.g., Influenza A, Influenza B, and parainfluenza), Papiloma virus, Papovaviridae, Paramyxoviridae, Prions, Parvoviridae, Phycodnaviridae, Picomaviridae (e.g. Rhinovirus, Poliovirus), Poxviridae (such as Smallpox or Vaccinia), Potyviridae, Reoviridae (e.g., Rotavirus), Retroviridae (HTLV-I, HTLV-II, Lentivirus), Rhabdoviridae, Tectiviridae, Togaviridae (e.g., Rubivirus), or any combination thereof. In another embodiment of the invention, the viral infection is caused by a virus selected from the group consisting of herpes, pox, papilloma, corona, influenza, hepatitis, sendai, sindbis, vaccinia viruses, west nile, hanta, or viruses which cause the common cold. In another embodiment of the invention, the condition to be treated is selected from the group consisting of AIDS, viral meningitis, Dengue, EBV, hepatitis, and any combination thereof.

Building and training an AbLM using publicly available unlabeled data to encode antibodies into embeddings or feature representations can enable various downstream applications, such as screening, prioritization, and/or design of broad-spectrum antibodies. Pre-training a pLM using a large set of unlabeled protein data (e.g., including millions of single-chain protein sequences) and initializing the AbLM from the pre-trained pLM enables the AbLM to be fine-tuned using a much smaller set of unlabeled antibody data (e.g., including thousands of paired VH and VL chain sequences), thereby overcoming one of the small data challenges discussed above where publicly available unlabeled antibody data are more limited than publicly available unlabeled protein data. Furthermore, using an AbLM, followed by a predictor (or activity prediction model) to predict efficacy of antibodies in neutralizing a certain virus enables the predictor to be trained using a substantially smaller set of labeled antibody data (e.g., including tens of antibody sequences with activity or response information), overcoming the other small data challenge discussed above where publicly available labeled antibody data is limited, especially during an early stage following the emergence of the virus. Masking CDR regions during AbLM training can increase emphasis on hypervariable, functionally relevant residues, thereby enhancing the AbLM's ability to predict or generate antibody variants with altered antigen-binding properties, and facilitating antibody design and redesign. Evaluating a newly designed or re-designed antibody via computational feedback or experimental testing and re-designing another antibody based on the evaluation (e.g., iterating in cycle) can generate an optimal antibody for a target virus. Updating or adapting parameters of the AbLM using reinforcement learning, for example, based on an evaluation from an antibody design and/or an evaluation from a predictor's output can enable the AbLM to continue to improve and adapt as new variants or new viruses emerge.

The AbLM discussed herein can be used to encode any type of antibodies (e.g., known antibodies, new antibodies, experimentally engineered antibodies, etc.) for downstream applications (e.g., antibody screening, prioritization, and/or design). The AbLM discussed herein can be used to design an antibody for treating a certain virus (e.g., a WT or variants). The AbLM discussed herein can enable antibody screening, prioritization, and/or design to be performed in a significantly less time than experimental screenings alone and can provide cost savings and reduce human efforts. Additionally, being able to screen and/or design antibodies quickly can be especially beneficial during an early stage following the emergence of a virus.

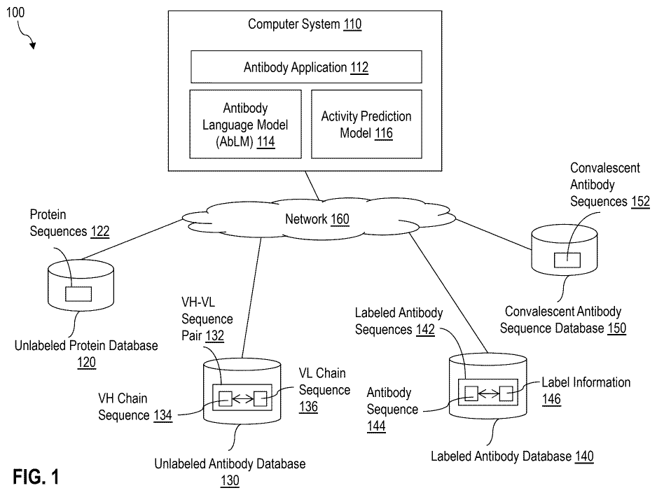

Turning now to FIG. 1, a network system 100 for implementing data-driven, ML-based screening, design, and/or prioritization of broad-spectrum antibodies is described. The network system 100 includes a computer system 110, an unlabeled protein domain database 120, an unlabeled antibody database 130, a labeled antibody database 140, a convalescent antibody database 150, and a network 160. The network 160 promotes communication between the components of the network system 100. The network 160 may be any communication network including a public data network (PDN), a public switched telephone network (PSTN), a private network, and/or a combination.

The unlabeled protein domain database 120 may be a repository of protein-related data, for example, storing protein sequences 122 without corresponding activity information. Each protein sequence 122 may be a sequence of amino acids, represented using standard amino acid alphabets. Each protein sequence 122 is a single-chain protein. In an example, the unlabeled protein domain database 120 may be a public database or library.

The unlabeled antibody database 130 may be a repository of antibody-related data, for example, storing variable heavy (VH)-variable light (VL) sequence pairs 132 without corresponding activity information. A VH-VL sequence pair 132 may include paired VH chain sequence 134 and VL chain sequence 136. A VH-VL sequence pair 132 may be part of an antibody. For instance, a full Y-shaped antibody may include two VH-VL sequence pairs 132. In an example, the unlabeled antibody database 130 may be a public database or library.

The labeled antibody database 140 may be a repository of antibody-related data with activity measurements, for example, storing labeled antibody sequences 142. Each labeled antibody sequence 142 may include an antibody sequence 144 and corresponding label information 146. The antibody sequence 144 may include a VH-VL sequence pair similar to the VH-VL sequence pair 132. The label information 146 may include annotations (e.g., characterizing profiles and/or activity against certain antigens or viruses). In an example, the labeled antibody database 140 may be a public database or library.

The convalescent antibody database 150 may be a repository of convalescent antibody-related data, for example, storing convalescent antibody sequences 152. The convalescent antibody sequences 152 are immune proteins collected from the blood plasma of people who have recovered from an infection (due to a certain WT and/or variants). Each convalescent antibody sequence 152 may include a VH-VL sequence pair similar to the VH-VL sequence pair 132.

In some instances, the protein sequences 122 in the unlabeled protein domain database 120 may also include a single VH chain sequence 134 and/or a single VL chain sequence 136 but not in paired form. While extensive protein data (e.g., the protein sequences 122) is available from public repositories, publicly available antibody data (e.g., the VH-VL sequence pairs 132) is relatively limited. Furthermore, publicly available activity data (e.g., the labeled antibody sequences 142) is even more limited. As an example, there may be millions of publicly accessible protein sequences 122, few thousands of publicly accessible protein VH-VL sequence pairs 132, and tens of publicly accessible labeled antibody sequences 142 for a particular virus (e.g., WT and its variants). As will be discussed more fully below, the present disclosure addresses the small data challenges by building an AbLM 114 in two stages: (1) pre-training using a large protein data set (e.g., from the unlabeled antibody database 130) and (2) fine-tuning using a smaller antibody data set (e.g., from the unlabeled antibody database 130), and building an activity prediction model 116 using a small activity data set (e.g., from the labeled antibody database 140).

The computer system 110 may include one or more computers. Computers are discussed further hereinafter. The computer system 110 may include an antibody application 112, an AbLM 114, and an activity prediction model 116. Each of the antibody application 112, the AbLM 114, and the activity prediction model 116 may include instructions stored in non-transitory transitory memory of the computer system 110 and executable by one or more processors of the computer system 100.

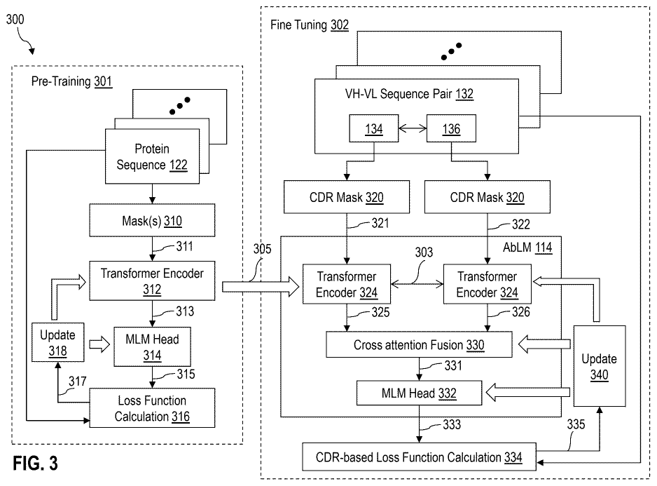

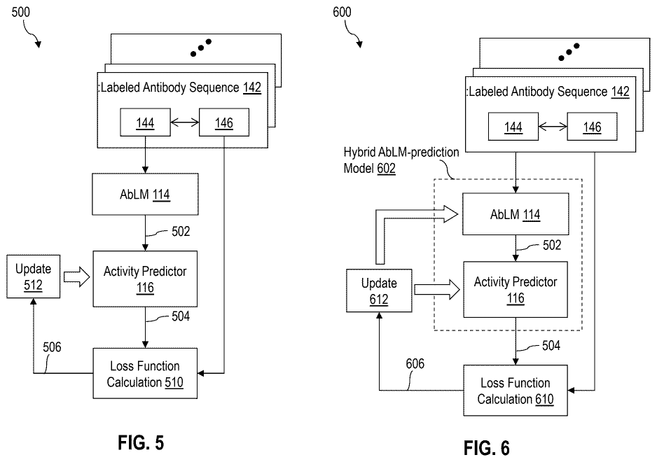

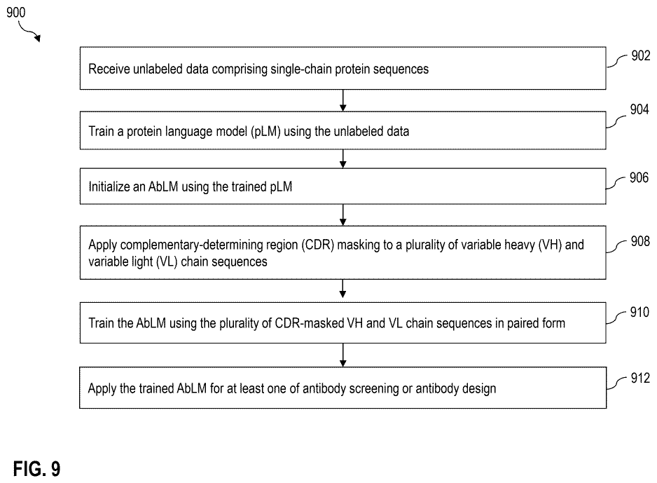

The antibody application 112 may train the AbLM 114 and the activity prediction model 116. To overcome the small data challenge discussed above, the antibody application 112 may build and train the AbLM 114 in part using unlabeled public data. According to an embodiment of the present disclosure, the antibody application 112 may build the AbLM 114 in part using one or more pLMs (e.g., the transformer encoders 312 of FIG. 3) trained on unlabeled protein sequence data (e.g., the protein sequences 122 from unlabeled protein domain database 120). Stated differently, the pLM may be used to warm start the AbLM 114. The antibody application 112 may fine-tune the AbLM 114 using unlabeled antibody sequence data (e.g., the VH-VL sequence pairs 132). The antibody application 112 may train an activity prediction model 116 for a particular virus (e.g., WT and its variants) using labeled antibody sequence data (e.g., the labeled antibody sequences 142 from the labeled antibody activity database 140). Mechanisms for training the AbLM 114 will be discussed more fully below with reference to FIGS. 3 and 9. Mechanisms for training the activity prediction model 116 will be discussed more fully below with reference to FIGS. 5 and 6.

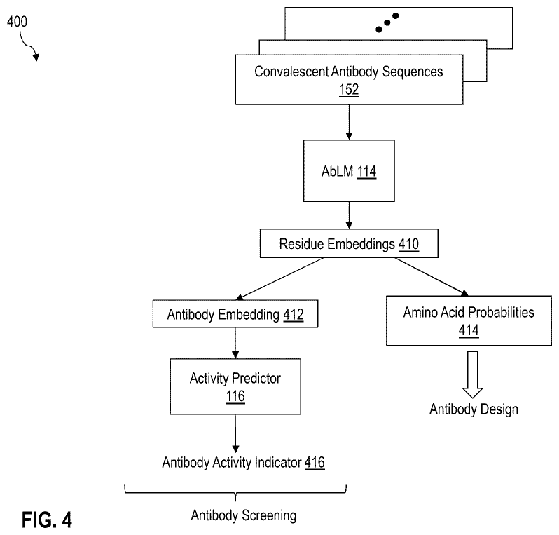

After the AbLM 114 and the activity prediction model 116 are trained, the antibody application 112 may use the AbLM 114 alone or in combination with the activity prediction model 116 for various downstream applications, such as antibody screening, design, and/or prioritization. Mechanisms for using the AbLM 114 will be discussed more fully below with reference to FIGS. 4, 7, and 9-11.

FIG. 1 is merely an example of components of a network system 100, and variations are contemplated to be within the scope of the present disclosure. In some embodiments, the network system 100 may include other components not illustrated in FIG. 1. In some embodiments, the network system 100 may not include every component illustrated in FIG. 1. In some embodiments, the components and communication links may be implemented with different communication links than those illustrated in FIG. 1. Generally, the unlabeled protein sequences 122, the unlabeled VH-VL sequence pairs 132, the labeled antibody sequences 142, and the convalescent antibody sequences 152 may be stored in any suitable number of databases and arranged in any suitable ways. Such and other embodiments are contemplated to be within the scope of the present disclosure.

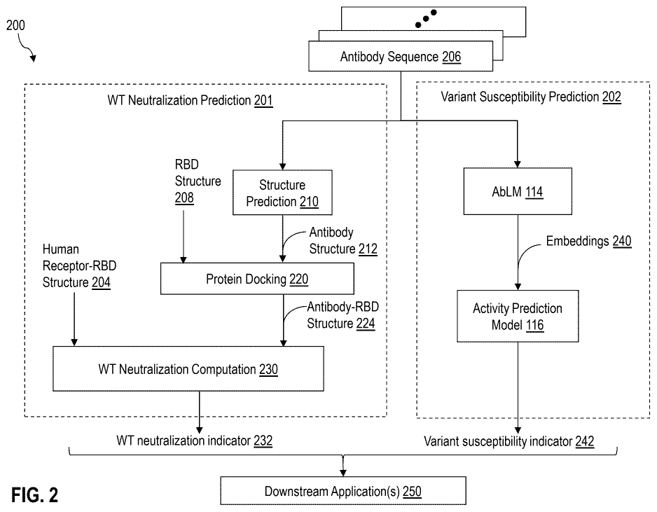

Turning now to FIG. 2, a framework 200 for analyzing broad-spectrum antibodies using physics-driven and data-driven, ML processes are described. The left side of FIG. 2 illustrates a WT neutralization prediction method 201. The right side of FIG. 2 illustrates a variant susceptibility (or neutralization) prediction method 202. The WT neutralization prediction method 201 and the variant susceptibility prediction method 202 may be implemented by the antibody application 112.

The WT neutralization prediction method 201 is physics-driven, or more specifically, structure prediction driven. Structure prediction identifies portions of the binding sites that are important based on structure proximity or energy calculations. The WT neutralization prediction method 201 predicts how WT proteins interact or bind to an antibody. Since WT proteins bind to the target (for instance, WT viral proteins bind to human proteins such as receptors), the WT neutralization prediction method 201 determines what portion of the target-binding residues, experimentally derived or computationally predicted (for instance, by structure prediction of the protein-protein complex), are blocked by the antibodies based on structure prediction. This determined portion, with possible varying forms of criteria on blockage and varying weights on individual binding residues, is then used as an indicator to predict WT neutralization, which is applicable even when no activity data is available for antibodies.

As shown in FIG. 2, the WT neutralization prediction method 201 may receive a set of antibody sequences 206 (e.g., from an antibody library). In an embodiment, the antibody sequences 206 may correspond to the VH-VL sequence pairs 132 in the unlabeled antibody database 130. Each antibody sequence 206 may be processed by a structure prediction module 210 and a protein docking module 220. The prediction module 210 and the protein docking module 220 may be computational physics-driven models. The prediction module 210 may generate an antibody structure 212 (e.g., a 3D antibody structure) for each respective antibody sequence 206. The protein docking module 220 may predict how the antibody structure 212 may interact (or bind) with a receptor binding domain (RBD) structure 208 (e.g., an antigenic domain of a spike protein) to provide an antibody-RBD structure 224. Stated differently, the protein docking module 220 may determine an interface or a binding-site between the antibody structure 212 and the RBD structure 208.

To determine the neutralization efficacy of the antibody sequence 206 against a target virus (e.g., having the RBD structure 208), the binding-site of the antibody-RBD structure 224 may be compared to the binding-site of a human receptor-RBD structure 204. The human receptor-RBD structure 204 may be obtained using various mechanisms. In some instances, a human receptor (e.g., angiotensin-converting enzyme 2 (ACE2) for SARS-CoV-2 virus) and an RBD may each be processed by the structure prediction module 210 to obtain respective human receptor and RBD structures, which may be further processed by the protein docking module 220 to obtain the human receptor-RBD structure 204. In other instances, the human receptor-RBD structure 204 may be computed from a human receptor-RBD complex. As shown, the antibody-RBD structure 224 and the human receptor-RBD structure 204 may be provided to the WT neutralization computation module 230. The WT neutralization computation module 230 may compute human receptor binding residue blocked by the antibody sequence 206 based on the antibody-RBD structure 224 and the human receptor-RBD structure 204. The WT neutralization computation module 230 may output a WT neutralization indicator 232 for a respective antibody sequence 206 against a particular WT under test. The WT neutralization indicator 232 may be indicative of various information, such as predicted binding free energy, structural interaction scores, and stability metrics of the antibody-antigen complex (e.g., the antibody-RBD structure 224), and the like. In an example, the WT neutralization indicator 232 may be a score indicative of neutralization efficacy of a respective antibody sequence 206 against a WT.

In some instances, the WT neutralization prediction method 201 may also provide structural basis for variant anticipation in addition to WT neutralization. Proteins, especially microbial proteins and cancer proteins, may evolve to weaken their interactions with the antibody (i.e., “escape” the antibody) under selective pressure, without weakening the interaction with their targets. Such antibody-resistant or antibody-escape variants may be anticipated from the structure prediction module 210 using geometry, energy, or ML predictions, which enables a proactive approach to broad-spectrum antibodies effective for WT, known variant, and anticipated variant proteins.

The variant susceptibility prediction method 202 is data-driven and ML-based. The variant susceptibility prediction method 202 uses an AbLM 114 and an activity prediction model 116 as disclosed herein. As will be discussed more fully below, the AbLM 114 may be built from one or more pLMs to perform various predictions and designs. In one embodiment, pLMs are used as encoders to represent an input antibody sequence (e.g., the antibody sequences 206, 142, 152, and/or paired VH chain sequence 134 and VL chain sequence 136) as embeddings 240 and connected in serial to the activity prediction model 116. The activity prediction model 116 may infer the antibody's neutralization activity against WT (experimentally measured or computationally inferred as discussed above) and activity against variants (known or anticipated as discussed above). For instance, the activity prediction model 116 may output a variant susceptibility indicator 242 for a respective antibody sequence 206 for a particular variant of the WT under test. In an example, the variant susceptibility indicator 242 may be a score indicative of susceptibility or neutralization efficacy of a respective antibody sequence 206 against a variant.

After processing all the antibody sequences 206 in the set, a WT neutralization indicator 232 and a variant susceptibility indicator 242 may be obtained for each of the antibody sequences 206. The WT neutralization indicators 232 and the variant susceptibility indicators 242 may be provided to one or more downstream applications 250. For instance, a first downstream application 250 may be for antibody screening, where the efficacy of an antibody sequence in neutralizing WT and/or variant(s) may be inferred by the WT neutralization indicators 232 and the variant susceptibility indicators 242. A second downstream application 250 may be for antibody prioritization, where the set of antibody sequences 206 may be ranked and prioritized based on the respective WT neutralization indicators 232 and the variant susceptibility indicators 242 against a broad-spectrum of viral variants. The ranking and prioritization may facilitate selection of one or more antibody sequences 206 that have the highest clinical potential for improvement (e.g., which may be suitable for designing a new antibody for treating a virus). A third downstream application 250 may be for antibody design or redesign. For instance, a new antibody can be generated based on one or more of the antibody sequences 206, and the WT neutralization prediction method 201 and the variant susceptibility prediction method 202 may be re-applied to assess the efficacy of the designed or modified antibody sequence in neutralizing the WT and/or the variants. Generally, the antibody redesign may iterate with experimental and/or computational feedback.

FIG. 2 is merely an example of a framework 200 for WT neutralization and/or variants susceptibility predictions, and variations are contemplated to be within the scope of the present disclosure. In some embodiments, the framework 200 may include other components not illustrated in FIG. 2. In some embodiments, the framework 200 may not include every component illustrated in FIG. 2. In some embodiments, the components and connections may be implemented with different connections than those illustrated in FIG. 2. For example, in some instances, the activity prediction model 116 may receive structural information of an antibody sequence 206 in addition to the embeddings 240 and may predict a susceptibility or neutralization efficacy of the antibody sequence 206 based on both inputs. Such and other embodiments are contemplated to be within the scope of the present disclosure.

Turning now to FIG. 3, an example method 300 of training an AbLM 114 to enable screening, prioritization, and/or design of broad-spectrum antibodies is described. The method 300 may be implemented by the antibody application 112. The method 300 trains the AbLM 114 using self-supervision techniques based on masked language modeling. The training includes two stages, a pre-training stage 301 shown on the left side of FIG. 3 and a fine-tuning stage 302 shown on the right side of FIG. 3. In the pre-training 301, a pLM is built using unlabeled data such as protein sequences 122 or protein structures (including antibody sequences or structures). In the illustrated example of FIG. 3, the pLM is a transformer encoder 312. At least some of the unlabeled data may be received from one or more public databases (e.g., the unlabeled protein database 120). The transformer encoder 312 may be pre-trained using publicly available protein sequences (e.g., the protein sequences 122). One embodiment of the pLM is pretrained on single polypeptide chains or single domains.

As shown in FIG. 3, the pre-training 301 may train a transformer encoder 312 using unlabeled protein sequences 122 (e.g., non-redundant protein domain sequences). At a high level, a portion of a protein sequence 122 may be masked or blanked-out, and the transformer encoder 312 may be trained to predict the amino acid(s) in the blanked-out portion. The amino acids at the masked position of the protein sequence 122 may function as the ground truth for the training. The transformer encoder 312 may include a plurality of stacked layers that include self-attention, feed-forward networks, residual connections, and normalization, producing contextualized sequence embeddings. The transformer encoder 312 may include a set of parameters (e.g., weights and/or biases) in each layer.

The pre-training 301 may apply a mask 310 to a protein sequence 122. The mask 310 may mask or blank out a portion of the protein sequence 122 to provide a masked protein sequence 311. The masked protein sequence 311 may be provided to the transformer encoder 312 to generate an embedding 313 (representing the protein sequence 122 in a latent space). The embedding 313 may be provided to an MLM head 314 or projector to generate amino acid probabilities 315 (e.g., a probability distribution over amino acids) at each sequence position. A loss function calculation 316 may be applied to the amino acid probabilities 315 for each sequence position and the original protein sequence 122 (before applying the mask 310). The loss function calculation 316 may calculate an error measurement 317 between the predicted amino acid at each blanked-out sequence position (by the mask 310) and the original amino acid at a corresponding sequence position in the original protein sequence 122. In an example, the loss function calculation 316 may compute a cross-entropy loss over masked positions, comparing the predicted probability distribution over tokens with a true token (e.g., representing the actual amino acid in the input sequence) at each masked position. The error measurement 317 may be provided to the update module 318. The update module 318 may adapt the parameters (e.g., weights and/or biases) of the transformer encoder 312 and the parameters of the MLM head 314 based on the error measurement 317. In an embodiment, the parameters may be updated using backward propagation and gradient algorithms. The pre-training 301 may be repeated over multiple iterations for the protein sequence 122, for example, until the error measurement 317 satisfies certain criteria (e.g., below a certain threshold) and may be further repeated for each of the protein sequences 122.

As further shown in the fine-tuning 302, the AbLM 114 may include two transformer encoders 324. The two transformer encoders 324 are identical (e.g., having an identical architecture and identical parameters). As such, the two transformer encoders 324 may be referred to as weight-tied as shown by the arrow 303. The fine-tuning 302 may initialize the two weight-tied transformer encoders 324 using the pre-trained transformer encoder 312 from the pre-training 301 (e.g., to “warm start” the fine-tuning 302). Stated differently, the fine-tuning 302 may start with two transformer encoders 324 with parameters initialized with values of respective parameters of the pre-trained transformer encoder 312 as shown by the arrow 305. The fine-tuning 302 may train or adapt the AbLM 114 using paired VH chain sequence 134 and VL chain sequence 136 (from the unlabeled antibody database 130). Each of the VH chain sequence 134 and VL chain sequence 136 in a VH-VL sequence pair 132 may be separately processed, each by a respective transformer encoder 324. At a high level, a portion of a VH chain sequence 134 and/or a portion of VL chain sequence 136 may be masked or blanked-out, and the transformer encoders 312 may be trained to predict the amino acid(s) in the blanked-out portion.

The fine-tuning 302 may apply a CDR mask 320 to each of the VH chain sequence 134 and VL chain sequence 136. CDRs are immunoglobulin hypervariable domains that determine specific antibody binding pattern towards antigens. Generally, the fine-tuning 302 may use any type of inductive bias masking strategy to fine-tune and/or train the AbLM 114. Example masking strategies may include, but are not limited to, a “vanilla” strategy, a “CDR-vanilla” strategy, a “CDR-margin” strategy, and a “CDR-pair” strategy. The “vanilla” strategy may apply a masking strategy that is used in bidirectional encoder representations from transformers (BERT) over the whole VH or VL chain sequences. For example, in one embodiment, 15% of amino acids may be masked, among those, 80% of amino acids may be replaced with <MASK> token, 10% of amino acids may be replaced by other random amino acids, and 10% of amino acids may remain unchanged. The “CDR-vanilla” strategy may apply the aforementioned BERT masking strategy in six CDR regions. The “CDR-margin” strategy may randomly select one CDR region (out of six) and mask all amino acids within the selected CDR region. The “CDR-pair” strategy may mask all amino acids in one pair of VH-VL CDR regions randomly selected among six CDR regions. In various embodiments, a number of potential additional masking techniques or combinations of techniques may be used. For example, 5% of amino acids outside CDR regions may be masked for any of the CDR biased strategies to enable density estimation of fairly constant framework regions. Generally, the fine-tuning 302 may apply the same CDR mask 320 (the same CDR strategy) or different CDR masks 320 (different CDR strategies) to the pair of the VH chain sequence 134 and the VL chain sequence 136.

After applying the CDR masks 320, the fine-tuning 302 may input the pair of the CDR-masked VH chain sequence 321 and CDR-masked VL chain sequence 322 separately into respective weight-tied transformer encoders 324 to generate corresponding VH embedding 325 and VL embedding 326. The VH embedding 325 and the VL embedding 326 are feature vectors representing the corresponding the VH chain sequence 134 and the VL chain sequence 136 in a latent space.

After generating the VH embedding 325 and the VL embedding 326, the fine-tuning 302 may apply a cross-attention fusion model 330 to the VH embedding 325 and the VL embedding 326. In an example, the cross-attention fusion model 330 may include bidirectional transformer encoders (e.g., with 12 layers and 12 heads per layer). The cross-attention fusion model 330 may allow information to flow between the two chains (e.g., the VH embedding 325 and the VL embedding 326). Stated differently, the cross-attention fusion model 330 may capture pairwise dependencies and structural relationships between the pair of VH chain sequence 134 and VL chain sequence 136. The cross-attention fusion model 330 may update the VH embedding 325 based on the VL embedding 326 and/or update the VL embedding 326 based on the VH embedding 325. The cross-attention fusion model 330 may output an antibody embedding 331 including a concatenation of the updated VH embedding 325 with the updated VL embedding 326.

After applying the cross-attention fusion model 330, the fine-tuning 302 may apply an MLM head 332 (e.g., a projector) to the antibody embedding 331 to generate an output including amino acid probabilities 333 (e.g., a probability distribution over amino acids) at each sequence position (of the VH chain sequence 134 and the VL chain sequence 136).

The fine-tuning 302 may evaluate the output of the AbLM 114. For instance, a CDR-based loss function calculation 334 may be applied to the amino acid probabilities 333 for each sequence position and the original input the VH chain sequence 134 and the VL chain sequence 136 (before applying the mask 310). The CDR-based loss function calculation 334 may calculate an error measurement 335 between the predicted amino acid at each blanked-out sequence position (by the CDR mask 320) and the original amino acid at a corresponding sequence position in the original input the VH chain sequence 134 and the VL chain sequence 136. In an example, the CDR-based loss function calculation 334 may compute a cross-entropy loss over CDR-masked positions, comparing the predicted probability distribution over tokens with a true token (e.g., representing the actual amino acid in the input sequence) at each masked position. The error measurement 335 may be provided to the update module 340. The update module 340 may adapt the parameters (e.g., weights and/or biases) of the transformer encoder 324, the parameters (e.g., weights and/or biases) of the cross-attention fusion model 330, and the parameters (e.g., weights and/or biases) of the MLM head 332 based on the error measurement 335. Since the two transformer encoders 324 are weight-tied, the parameters remain identical after the update. In an embodiment, the parameters of the transformer encoders 324, the cross-attention fusion model 330, and the MLM head 332 may be updated using backward propagation and gradient algorithms. The fine-training 302 may be repeated over multiple iterations for the input of the VH chain sequence 134 and the VL chain sequence 136 until the error measurement 335 satisfies certain criteria (e.g., below a certain threshold) and may be further repeated for each of the VH-VL sequence pairs 132. In an example, the convergence determination for the training may be based on a perplexity measure or a top-1 accuracy measure.

Turning now to FIG. 4, an example method 400 of using an AbLM 114 for screening, design, and/or prioritization of broad-spectrum antibodies is described. The method 400 may be implemented by the antibody application 112. The AbLM 114 may be trained using the method 300 discussed above with reference to FIG. 3. As shown in FIG. 4, the AbLM 114 may receive a plurality of convalescent antibody sequences 152 as input. Each convalescent antibody sequence 152 may include a paired VH and VL chain sequence similar to the VH-VL sequence pair 132. In an example, the convalescent antibody sequences 152 may correspond to the antibody sequences 206. The AbLM 114 may process each convalescent antibody sequence 152 to generate residue embeddings 410, an antibody embedding 412, and/or amino acid probabilities 414.

The residue embeddings 410 and the antibody embeddings 412 may be output from an intermediate layer of the AbLM 114 (e.g., from the cross-attention fusion model 330 of the AbLM 114). The residue embeddings 410 may include residue embeddings corresponding to each residue of the VH chain sequence 134 and the VL chain sequence 136 in the respective input convalescent antibody sequence 152. Each residue embedding 410 may include a feature vector representation of a single amino acid at a respective sequence position. The antibody embedding 412 may be generated from respective chain-level embeddings of the VH chain sequence 134 and the VL chain sequence 136. Each chain-level embedding may be obtained by mean pooling residue embeddings 410 corresponding to the respective chain sequence to produce a fixed-dimensional feature vector. In some embodiments, each of the VH and VL chain-level embeddings may be a 768-dimensional vector. The antibody sequence-level embedding may be formed by concatenating the VH chain-level embedding and the VL chain-level embedding to produce, for example, a 1536-dimensional antibody embedding. In some instances, the antibody sequence-level embedding may include inserting padding tokens to generate a 1536-dimensional antibody embedding as the VH chain sequences 134 and the VL chain sequences 136 may have variable lengths. For instance, the padding may be based on the chain sequence with the longest length. The amino acid probabilities 414 may be output from a final layer of the AbLM 114 (e.g., from the MLM head 332 of the AbLM 114). For instance, the MLM head 332 may transform the residue embeddings 410 into amino acid logits (unnormalized probabilities) per residue, followed by normalizing the amino acid probabilities per residue via a “softmax” function. The amino acid probabilities 414 is provided for each sequence position. In other words, the amino acid probabilities 414 may include a probability distribution over amino acids at each sequence position of each of the VH chain sequence (e.g., the VH chain sequence 134) and the VL chain sequence (e.g., the VL chain sequence 136) in the input convalescent antibody sequence 152.

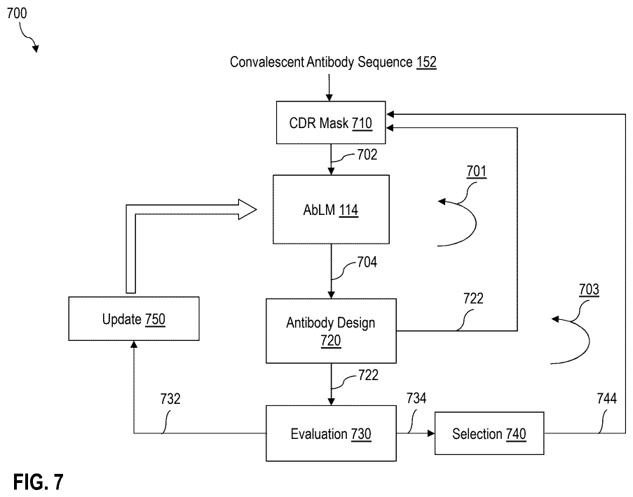

The outputs of the AbLM 114 may be used for antibody screening (in the left branch) and/or antibody design (in the right branch). For antibody screening, the antibody embedding 412 (for a corresponding convalescent antibody sequence 152) may be input to an activity prediction model 116. The activity prediction model 116 may be trained to predict activity associated with a particular virus (e.g., WT and its variants). The training will be discussed more fully below with reference to FIGS. 5 and 6. As shown in FIG. 4, the activity prediction model 116 may output an antibody activity indicator 416 (e.g., corresponding to the variant susceptibility indicator 242) for an input convalescent antibody sequence 152. The activity indicator 416 may indicate the susceptibility or neutralization efficacy of the input convalescent antibody sequence 152 against the particular virus or variant. The activity prediction model 116 may be a nonparametric Gaussian regressor such as Kriging. Kriging uses few parameters, which may be beneficial and assist in addressing the small data challenge discussed above. There are also efficiencies gained by using a simpler ML model such as Kriging (e.g., processing efficiencies, memory efficiencies, etc.). However, any suitable ML models and/or computational biology techniques may be used for the activity prediction model 116. For antibody design (or redesign), the amino acid probabilities 414 may be sampled to redesign or generate a new antibody sequence. An example of antibody design (or redesign) will be discussed below with reference to FIG. 7.

While FIG. 4 is illustrated with the AbLM 114 receiving the convalescent antibody sequences 152 as input for antibody screening and/or design, the AbLM 114 may generally be applied to any suitable antibody sequences (e.g., known antibodies, new antibodies, experimentally engineered antibodies, etc.) for antibody screening and/or design.

Turning now to FIG. 5, an example method 500 of training an activity prediction model 116 to predict activity for a target virus is described. The method 500 may be implemented by the antibody application 112. The activity prediction model 116 may be trained using labeled antibody sequences 142 (e.g., from the labeled antibody database 140). As discussed above, each labeled antibody sequence 142 may include an antibody sequence 144 including a pair of VH chain sequence (e.g., VH chain sequence 134) and VL chain sequence (e.g., VL chain sequence 136) and corresponding label information 146 characterizing profiles and/or activity against a certain antigen or virus indicating activities or responses associated with the antibody sequence 144.

The method 500 may train the activity prediction model 116 with the parameters (e.g., weights and/or biases) of the AbLM 114 being fixed, for example, after the AbLM 114 is trained using the method 300 discussed above with reference to FIG. 3. To train the activity prediction model 116, the AbLM 114 may process each antibody sequence 144 in the labeled antibody sequence 142 to generate an antibody embedding 502 (e.g., similar to the antibody embedding 412) representing the antibody sequence 144 in a latent space. The activity prediction model 116 may process the antibody embedding 502 to generate an antibody activity indicator 504 (e.g., similar to the antibody activity indicator 416). Next, a loss function calculation 510 may be applied to the antibody activity indicator 504 and the label information 146 corresponding to the input antibody sequence 144 to calculate an error measurement 506 between the antibody activity indicator 504 and the label information 146. The error measurement 506 may be provided to the update module 512. The update module 512 may adapt the parameters (e.g., weights and/or biases) of the activity prediction model 116 based on the error measurement 506. In an embodiment, the parameters may be updated using backward propagation and gradient algorithms. The AbLM 114 and the activity prediction model 116 processing may be repeated over multiple iterations for the labeled antibody sequence 142 until the error measurement 506 satisfies certain criteria (e.g., below a certain threshold) and may be further repeated for each of the labeled antibody sequence 142.

Turning now to FIG. 6, an example method 600 of training a hybrid AbLM-prediction model 602 to predict antibody activity for a target virus is described. The method 600 may be implemented by the antibody application 112. The hybrid AbLM-prediction model 602 includes the AbLM 114, followed by the activity prediction model 116 in serial connection with the AbLM 114. The method 600 is substantially similar to the method 500. For instance, the AbLM 114 and the activity prediction model 116 may process each input labeled antibody sequence 142, and a loss function calculation 610 may be applied using similar mechanisms as the loss function calculation 510 as discussed above with reference to FIG. 5. However, in the method 600, the AbLM 114 and the activity prediction model 116 are jointly trained using the labeled antibody sequences 142. More specifically, the AbLM 114 in the hybrid AbLM-prediction model 602 may operate as a fine-tunable encoder trained or adapted together with the activity prediction model 116.

As shown in FIG. 6, an update module 612 may update parameters (e.g., weights and/or biases) of the AbLM 114 and the parameters (e.g., weights and/or biases) of the activity prediction model 116 based on the error measurement 606 output by the loss function calculation 610. In an embodiment, the update module 612 may apply backward propagation and gradient algorithms to adapt each of the AbLM 114 and activity prediction model 116. The training of the hybrid AbLM-prediction model 602 may be iterated as discussed above with reference to FIG. 5.

The activity prediction model 116 operating as a prediction head may use much less parameters compared to the encoder (e.g., the AbLM 114). Generally, the smaller size activity prediction model 116 (with less model parameters) can be trained using a significantly smaller data set than the larger size AbLM 114 (with more model parameters). As discussed above, publicly accessible labeled antibody data is limited. Thus, training an AbLM 114 using pre-training 301 on a large unlabeled protein data set (e.g., including millions of protein sequences 122), followed by fine-tuning 302 on a smaller unlabeled antibody data set (e.g., including thousands of VH-VL sequence pairs 132), and further training an activity prediction model 116 using very few labeled antibody sequences 142 (e.g., tens of labeled antibody sequences 142) can address the small data challenges discussed above.

Turning now to FIG. 7, an example method 700 of using an AbLM 114 to design broad-spectrum antibodies for a target virus is described. The method 700 may be implemented by the antibody application 112. The AbLM 114 may be trained using the method 300 discussed above with reference to FIG. 3. As shown in FIG. 7, the AbLM 114 may receive a convalescent antibody sequence 152 as input. Each convalescent antibody sequence 152 may include a paired VH and VL chain sequence similar to the VH-VL sequence pair 132. A CDR mask 710 may mask a portion (e.g., a CDR) of the convalescent antibody sequence 152. The CDR mask 710 may be substantially similar to the CDR mask 320 and may use any one of the CDR masking strategies discussed above or any other suitable masking. The AbLM 114 may process the CDR-masked antibody sequence 702 to generate amino acid probabilities 704 (e.g., a probability distribution over amino acids) at each sequence position (of a corresponding VH chain sequence and the VL chain sequence) similar to the amino acid probabilities 414 as discussed above with reference to FIG. 4.

The per-position amino acid probabilities 704 may be provided to an antibody design module 720. The antibody design module 720 may sample the per-position amino acid probabilities 704 to generate an antibody sequence 722 (e.g., a new or re-designed antibody sequence). The sampling may include selecting an amino acid at each sequence position (e.g., within a masked CDR region) based on the amino acid probabilities 704 at the respective sequence position based on certain criteria. In an example, the sampling may be based on a mutation sampling strategy, such as identifying the top-m positions with the highest amino-acid probability entropies where m~Poisson(μ)+1(subject to a trust radius; μ is a sequence mutation proposal rate) and sampling the top-K amino-acid types with the highest probabilities at each of the m positions. In an embodiment, the sampling may be iterated, for example, by feeding the generated or new antibody sequence 722 back to the CDR mask 710 and the AbLM 114 to generate another antibody sequence 722. In other words, the AbLM 114 may be used to assist in re-designing a new antibody sequence 722. The re-designing may generally be iterated one or more times (e.g., shown by the loop 701). In an example, the number of iterations may be based on a threshold or other criteria.

The antibody design module 720 may provide the generated antibody sequence 722 (after any suitable number of redesign iterations) to an evaluation module 730. The evaluation module 730 may evaluate the neutralization efficacy of the generated antibody sequence 722 against a target virus. In an example, the evaluation may include experimentally testing the generated antibody sequence 722. In an example, the evaluation may be ML-based, for example, using the method 400 (e.g., the left output branch) to generate an antibody activity indicator 416. In an example, the evaluation may be based on computational biology techniques. Generally, the evaluation module 730 can use any suitable combination of ML-based evaluation, experimental testing, or computational biology techniques.

In an embodiment, the antibody design module 720 can generate multiple antibody sequences 722 based on the sampling, and the evaluation module 720 may evaluate the neutralization efficacy of each of the generated antibody sequences 722 as discussed above. In such an embodiment, a selection model 740 can select one of the generated antibody sequences 722 and feed the selected antibody sequence 744 to the CDR mask 710. For instance, the evaluation module 730 can provide an output 734 including the generated antibody sequences 722 and corresponding neutralization efficacy to the selection module 740 for the selection. In an embodiment, the AbLM 114 can be retrained based on the evaluation result 732 outputted by the evaluation module 730. For instance, an update module 750 may adapt the parameters (e.g., weights and/or biases) of the AbLM 114 based on the evaluation result 732. In an embodiment, the update may be based on backward propagation and gradient algorithms.

Stated differently, for antibody redesign, a partially known antibody sequence (e.g., a CDR-masked antibody sequence 702) may be fed to the AbLM 114 for generating a new antibody sequence 722. The partially known antibody sequence may be a known antibody sequence (e.g., a convalescent antibody sequence 152) with a portion being masked (e.g., using one of the CDR masking strategies discussed above or any suitable masking template). The redesign may include iterative design loops 701 and/or 703 in which partially known sequences (e.g., CDR-masked antibody sequences 702) are used to guide the redesign (or sampling of amino acid probabilities 704). The redesign or sampling may include predicting amino acid probabilities 704 in masked CDRs, generating antibody candidates, and iterating with computational or experimental feedback (e.g., the evaluation 730). That is, the partially known sequences may evolve through a feedback loop between the input to the CDR mask 710 and the output of the antibody design module 720. In an example, the number of iterations for the design may be predetermined. In an example, an adaptive stopping rule may be applied to terminate the feedback loop, for example, when there is no significant improvement in new designs or when there is no significant changes in the designed sequences.

While FIG. 7 is illustrated with the AbLM 114 receiving the convalescent antibody sequences 152 as input for antibody design, the AbLM 114 may generally be applied to any suitable antibody sequences (e.g., known antibodies, new antibodies, experimentally engineered antibodies, etc.) for antibody design. Furthermore, an antibody sequence 722 generated by the antibody design module 720 may or may not iterate over the CDR mask 710 and AbLM 114 (e.g., the loop 701) before being evaluated by the evaluation module 730.

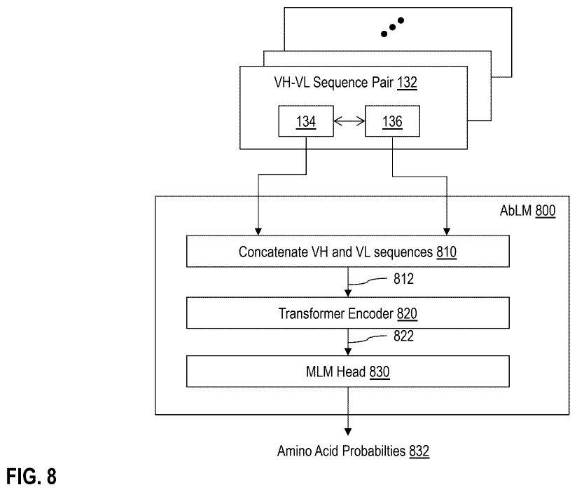

Turning now to FIG. 8, an AbLM 800 with sequence concatenation is described. The AbLM 800 may be implemented by the antibody application 112. In an embodiment, the AbLM 800 may be used in place of the AbLM 114 in the framework 200 and methods 300, 400, 500, 600, and 700 discussed above with reference to FIGS. 2, 3, 4, 5, 6, and 7, respectively. The AbLM 800 may be substantially similar to the AbLM 114. For instance, the AbLM 800 may receive a pair of the VH chain sequence 134 and the VL chain sequence 136 and generate embeddings 822 for the VH chain sequence 134 and the VL chain sequence 136. However, instead of processing a pair of the VH chain sequence 134 and the VL chain sequence 136 separately using two weight-tied transformer encoders 324 as in the AbLM 114, the AbLM 800 includes a VH and VL sequence concatenation module 810 and a single transformer encoder 820.

The VH and VL sequence concatenation module 810 may concatenate the pair of VH chain sequence 134 and the VL chain sequence 136 with a special token <SEP> into a single concatenated VH-VL sequence 812. A special token is a token that is not present in protein sequences and does not correspond to a specific amino-acid type. In an example, one or more special tokens may be used to concatenate the VH chain sequence 134 and the VL chain sequence 136, where the one or more tokens may be padding tokens <PAD> to change a variable-length sequence into a fixed-length sequence (with predetermined length). In an example, the concatenation may use two special amino acids (U and O), three ambiguity letters (X, B, and Z), and five special tokens including <PAD> and <SEP>.

The concatenation may enable the pair of the VH chain sequence 134 and the VL chain sequence 136 to be processed by a single pretrained pLM as a single sequence input. In the illustrated example of FIG. 8, the pretrained pLM is a transformer encoder 820. The transformer encoder 820 may be similar to the transformer encoder 324. The transformer encoder 820 may be trained to encode the concatenated VH-VL sequence 812 into embeddings 822 (e.g., a feature representation of the VH chain sequence 134 and the VL chain sequence 136 in a latent space). The embedding 822 may include residue embeddings and an antibody embedding similar to the residue embeddings 410 and the antibody embedding 412 discussed above with reference to FIG. 4. The AbLM 800 may further include an MLM head 830 similar to the MLM head 332. The MLM head 830 may be trained to generate amino acid probabilities 832 (e.g., a probability distribution over amino acids) at each sequence position based on the residue embeddings in the embeddings 822. The AbLM 800 may be trained in a substantially similar way as discussed above with reference to FIG. 3. For instance, the AbLM 800 may be initialized from a pLM pretrained as discussed above with reference to the pre-training 301 of FIG. 3 and fine-tuned on unlabeled antibody data (e.g., the VH-VL sequence pairs 132) with CDR masking as discussed above with reference to the fine-tuning 302 of FIG. 3. During an inference stage, the trained AbLM 800 may process an input antibody sequence (e.g., the antibody sequences 144, 152, or 206 or the VH-VL sequence pairs 132) to generate embeddings 822 and/or amino acid probabilities 832 (e.g., a probability distribution over amino acids) at each sequence position.

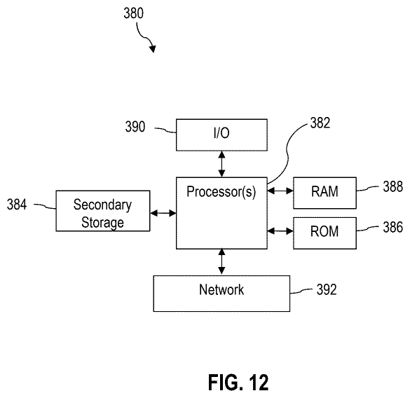

Turning now to FIG. 9, a method 900 is described. In an embodiment, the method 900 is a method of building and training an AbLM 114 or 800 to provide antibody screening or design for virus neutralization. The method 900 may include similar mechanisms as discussed above with reference to FIGS. 1-8. The method 900 may be implemented by an antibody application 112 including instructions stored in a non-transitory memory of a computer system 110 and executable by a processor of the computer system 110. In embodiments, the method 900 may be implemented using a computer system with components as shown in FIG. 12. As illustrated, FIG. 9 includes a number of enumerated operations, but embodiments of the operations in FIG. 9 may include additional operations before, after, and in between the enumerated operations. In some embodiments, one or more of the enumerated operations may be omitted or performed in a different order.

At operation 902, the antibody application 112 receives unlabeled data (e.g., the unlabeled protein database 120) comprising single-chain protein sequences (e.g., the protein sequences 122). At operation 904, the antibody application 112 trains a pLM (e.g., the transformer encoder 312) using the unlabeled data. At operation 906, the antibody application 112 initializes an AbLM 114 or 800 using the trained pLM.

At operation 908, the antibody application 112 applies CDR masking (e.g., the CDR masks 320) to a plurality of VH and VL chain sequences (e.g., the VH-VL sequence pair 132). In an embodiment, as part of applying the CDR masking, the antibody application 112 randomly selects one CDR region and masks all amino acids within the one CDR region.

At operation 910, the antibody application 112 trains the AbLM 114 or 800 using the plurality of CDR-masked VH and VL chain sequences 321 and 322 in paired form (e.g., as discussed above with reference to FIG. 3). In an embodiment, as part of training the AbLM 114 or 800, the antibody application 112 processes an individual pair of the CDR-masked VH and VL chain sequences 321 and 322 separately using respective ones of two identical (or weight-tied) pLMs (e.g., the transformer encoders 324) to generate corresponding VH embeddings 325 and VL embeddings 326. The two identical pLMs may have identical parameters and may be initialized using parameters of the pLM trained at operation 904. The antibody application 112 further applies a cross-attention fusion model 330 to the VH embeddings 325 and the VL embeddings 326. In an embodiment, as part of training the AbLM 114 or 800, the antibody application 112 processes a pair of the CDR-masked VH and VL chain sequences 321 and 322 using the AbLM 114 or 800 to generate an output. The antibody application 112 further adapts one or more parameters of the AbLM 114 or 800 based on an error measurement 335 between the output of the AbLM 114 or 800 and the pair of the CDR-masked VH and VL chain sequences 321 and 322.

At operation 912, the antibody application 112 applies the trained AbLM 114 or 800 for at least one of antibody screening or antibody design (e.g., as discussed above with reference to FIGS. 2, 4, and 7). In an embodiment, as part of applying the trained AbLM 114 or 800, the antibody application 112 provides an antibody sequence (e.g., the VH-VL sequence pairs 132, the antibody sequences 142, 152, or 206) including a VH chain sequence 134 and a VL chain sequence 132 as an input to the trained AbLM 114 or 800. The antibody application 112 further receives, from the trained AbLM 114 or 800, an output including at least one of embeddings 410 and/or 412 representative of the antibody sequence or a probability distribution of amino acids at each sequence position (e.g., the per-position amino acid probabilities 414 or 704) of the antibody sequence.

In an embodiment, as part of applying the trained AbLM 114 or 800 for the antibody screening at operation 912, the antibody application 112 further predicts at least one activity for the antibody sequence based on the output of the trained AbLM 114 or 800. In an embodiment, predicting the at least one activity for the antibody sequence is based on an activity prediction model 116 trained using labeled data (e.g., the labeled antibody database 140) including antibody sequences 144 and corresponding label information 146 indicative of response activities associated with a target virus (e.g., as discussed above with reference to FIGS. 2 and 4).

In an embodiment, as part of applying the trained AbLM 114 or 800 for the antibody design at operation 912, the antibody application 112 generates a second antibody sequence 722 based on sampling one or more probability distributions of amino acids at one or more sequence positions of the antibody sequence (e.g., as discussed above with reference to FIG. 7). In an embodiment, the antibody application 112 further retrains the AbLM 114 or 800 using at least one of experimental feedback from experiments or reinforcement learning (e.g., the evaluation 730) based on the output from the trained AbLM 114 or 800.

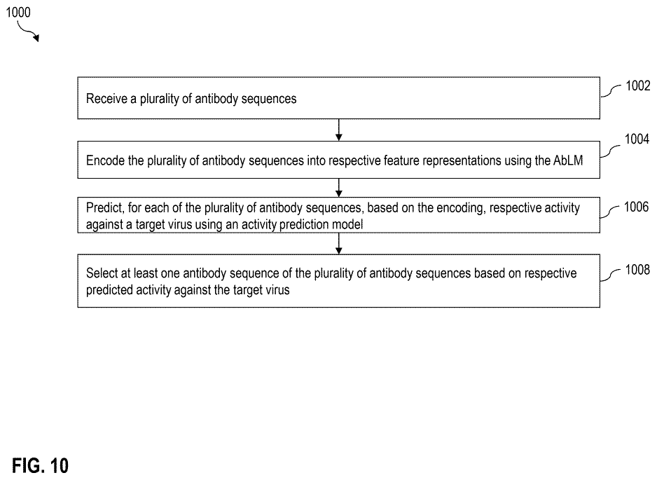

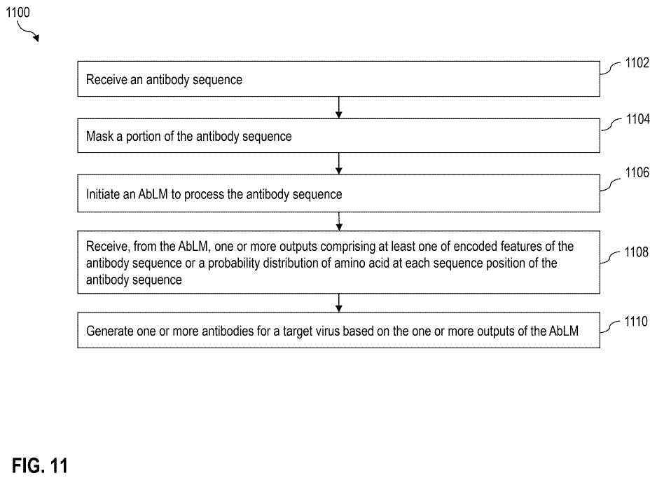

Turning now to FIG. 10, a method 1000 is described. In an embodiment, the method 1000 is a method of performing ML-based antibody selection for treating a target virus using an AbLM 114 or 800. The method 1000 may include similar mechanisms as discussed above with reference to FIGS. 1-9. The method 1000 may be implemented by an antibody application 112 including instructions stored in a non-transitory memory of a computer system 110 and executable by a processor of the computer system 110. In embodiments, the method 1000 may be implemented using a computer system with components as shown in FIG. 12. As illustrated, FIG. 10 includes a number of enumerated operations, but embodiments of the operations in FIG. 10 may include additional operations before, after, and in between the enumerated operations. In some embodiments, one or more of the enumerated operations may be omitted or performed in a different order.

At operation 1002, the antibody application 112 receives a plurality of antibody sequences (e.g., the antibody sequences 144, 152, or 206 or the VH-VL sequence pairs 132). At operation 1004, the antibody application 112 encodes the plurality of antibody sequences into respective feature representations (e.g., the embeddings 240, 331, 412) using the AbLM 114 or 800.

At operation 1006, the antibody application 112 predicts, for each of the plurality of antibody sequences, based on the encoding at operation 1004, respective activity against a target virus using an activity prediction model 116. In an embodiment, the AbLM 114 or 800 is trained using unlabeled data (e.g., the unlabeled protein database 120 and/or the unlabeled antibody database 130) including at least one of protein sequences 122 or antibody sequences 132 and the activity prediction model 116 is trained using first labeled data (e.g., the labeled antibody database 140) including first antibody sequences 144 and respective first activity information 146 associated with a target virus. In an embodiment, the AbLM 114 or 800 and the activity prediction model 116 are jointly trained using second labeled data (e.g., the labeled antibody database 140) including second antibody sequences 144 and respective second activity information 146 associated with a target virus.

At operation 1008, the antibody application 112 selects at least one antibody sequence of the plurality of antibody sequences based on respective predicted activity against the target virus. In an embodiment, the selected at least one antibody sequence is experimentally tested against the target virus. In an embodiment, the selected at least one antibody sequence is present in a composition further including a pharmaceutically acceptable carrier or excipient. In an embodiment, the antibody application 112 further adapts one or more parameters of the AbLM 114 or 800 based on results of experimentally testing the selected at least one antibody sequence against the target virus.

Turning now to FIG. 11, a method 1100 is described. In an embodiment, the method 1100 is a method of using an AbLM 114 or 800 for antibody design. The method 1100 may include similar mechanisms as discussed above with reference to FIGS. 1-10. The method 1100 may be implemented by an antibody application 112 including instructions stored in a non-transitory memory of a computer system 110 and executable by a processor of the computer system 110. In embodiments, the method 1100 may be implemented using a computer system with components as shown in FIG. 12. As illustrated, FIG. 11 includes a number of enumerated operations, but embodiments of the operations in FIG. 11 may include additional operations before, after, and in between the enumerated operations. In some embodiments, one or more of the enumerated operations may be omitted or performed in a different order.

At operation 1102, the antibody application 112 receives an antibody sequence (e.g., the antibody sequences 144, 152, or 206 or the VH-VL sequence pairs 132). In an embodiment, the antibody sequence includes an experimentally generated antibody sequence. At operation 1104, the antibody application 112 masks a portion of the antibody sequence. In an embodiment, masking the portion of the antibody sequence is based on CDR masking (e.g., using the CDR mask 310 or 710).

At operation 1106, the antibody application 112 initiates an AbLM 114 or 800 to process the antibody sequence. At operation 1108, the antibody application 112 receives, from the AbLM 114 or 800, one or more outputs including at least one of encoded features (e.g., the embeddings 240, 331, 412) of the antibody sequence or a probability distribution of amino acid at each sequence position (e.g., the per-position amino acid probabilities 414 or 704) of the antibody sequence.

At operation 1110, the antibody application 112 generates one or more antibodies for a target virus based on the one or more outputs of the AbLM 114 or 800. In an embodiment, generating the one or more antibodies for the target virus is further based on sampling one or more probability distributions of amino acids at one or more sequence positions within the masked portion of the antibody sequence. In an embodiment, at least one of the one or more generated antibodies is present in a composition further comprising a pharmaceutically acceptable carrier or excipient. In an embodiment, the antibody application 112 further adapts one or more parameters of the AbLM 114 or 800 based on experimentally testing the one or more designed antibodies against the target virus.

FIG. 12 illustrates a computer system 380 suitable for implementing one or more embodiments disclosed herein. The computer system 380 includes one or more processors 382 that are in communication with memory devices including secondary storage 384, read only memory (ROM) 386, RAM 388, input/output (I/O) devices 390, and network connectivity devices 392. The processor(s) 382 may be implemented as one or more central processing unit (CPU) chips and/or one or more graphical processing unit (GPU) chips.

It is understood that by programming and/or loading executable instructions onto the computer system 380, at least one of the processor 382, the RAM 388, and the ROM 386 are changed, transforming the computer system 380 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an ASIC that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 380 is turned on or booted, the processor(s) 382 may execute a computer program or application. For example, the processor(s) 382 may execute software or firmware stored in the ROM 386 or stored in the RAM 388. In some cases, on boot and/or when the application is initiated, the processor(s) 382 may copy the application or portions of the application from the secondary storage 384 to the RAM 388 or to memory space within the processor(s) 382 itself, and the processor(s) 382 may then execute instructions that the application is comprised of. In some cases, the processor(s) 382 may copy the application or portions of the application from memory accessed via the network connectivity devices 392 or via the I/O devices 390 to the RAM 388 or to memory space within the processor(s) 382, and the processor(s) 382 may then execute instructions that the application is comprised of. During execution, an application may load instructions into the processor(s) 382, for example load some of the instructions of the application into a cache of the processor(s) 382. In some contexts, an application that is executed may be said to configure the processor(s) 382 to do something, e.g., to configure the processor(s) 382 to perform the function or functions promoted by the subject application. When the processor(s) 382 are configured in this way by the application, the processor(s) 382 become a specific purpose computer or a specific purpose machine.

The secondary storage 384 is typically comprised of one or more disk drives or tape drives and is used for non-volatile storage of data and as an over-flow data storage device if RAM 388 is not large enough to hold all working data. Secondary storage 384 may be used to store programs which are loaded into RAM 388 when such programs are selected for execution. The ROM 386 is used to store instructions and perhaps data which are read during program execution. ROM 386 is a non-volatile memory device which typically has a small memory capacity relative to the larger memory capacity of secondary storage 384. The RAM 388 is used to store volatile data and perhaps to store instructions. Access to both ROM 386 and RAM 388 is typically faster than to secondary storage 384. The secondary storage 384, the RAM 388, and/or the ROM 386 may be referred to in some contexts as computer readable storage media and/or non-transitory computer readable media.

I/O devices 390 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 392 may take the form of modems, modem banks, Ethernet cards, USB interface cards, serial interfaces, token ring cards, fiber distributed data interface (FDDI) cards, wireless local area network (WLAN) cards, radio transceiver cards, and/or other well-known network devices. The network connectivity devices 392 may provide wired communication links and/or wireless communication links (e.g., a first network connectivity device 392 may provide a wired communication link and a second network connectivity device 392 may provide a wireless communication link). Wired communication links may be provided in accordance with Ethernet (IEEE 802.3), Internet protocol (IP), time division multiplex (TDM), data over cable service interface specification (DOCSIS), wavelength division multiplexing (WDM), and/or the like. In an embodiment, the radio transceiver cards may provide wireless communication links using protocols such as CDMA, global system for mobile communications (GSM), LTE, WiFi (IEEE 802.11), Bluetooth, Zigbee, narrowband Internet of things (NB IoT), near field communications (NFC), and radio frequency identity (RFID). The radio transceiver cards may promote radio communications using 5G, 5G New Radio, or 5G LTE radio communication protocols. These network connectivity devices 392 may enable the processor 382 to communicate with the Internet or one or more intranets. With such a network connection, it is contemplated that the processor 382 might receive information from the network, or might output information to the network in the course of performing the above-described method steps. Such information, which is often represented as a sequence of instructions to be executed using processor 382, may be received from and outputted to the network, for example, in the form of a computer data signal embodied in a carrier wave.

Such information, which may include data or instructions to be executed using processor 382 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well-known to one skilled in the art. The baseband signal and/or signal embedded in the carrier wave may be referred to in some contexts as a transitory signal.

The processor 382 executes instructions, codes, computer programs, scripts which it accesses from hard disk, floppy disk, optical disk (these various disk-based systems may all be considered secondary storage 384), flash drive, ROM 386, RAM 388, or the network connectivity devices 392. While only one processor 382 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 384, for example, hard drives, floppy disks, optical disks, and/or other device, the ROM 386, and/or the RAM 388 may be referred to in some contexts as non-transitory instructions and/or non-transitory information.

In an embodiment, the computer system 380 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 380 to provide the functionality of a number of servers that is not directly bound to the number of computers in the computer system 380. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the computer system 380, at least portions of the contents of the computer program product to the secondary storage 384, to the ROM 386, to the RAM 388, and/or to other non-volatile memory and volatile memory of the computer system 380. The processor 382 may process the executable instructions and/or data structures in part by directly accessing the computer program product, for example by reading from a CD-ROM disk inserted into a disk drive peripheral of the computer system 380. Alternatively, the processor 382 may process the executable instructions and/or data structures by remotely accessing the computer program product, for example by downloading the executable instructions and/or data structures from a remote server through the network connectivity devices 392. The computer program product may comprise instructions that promote the loading and/or copying of data, data structures, files, and/or executable instructions to the secondary storage 384, to the ROM 386, to the RAM 388, and/or to other non-volatile memory and volatile memory of the computer system 380.

In some contexts, the secondary storage 384, the ROM 386, and the RAM 388 may be referred to as a non-transitory computer readable medium or a computer readable storage media. A dynamic RAM embodiment of the RAM 388, likewise, may be referred to as a non-transitory computer readable medium in that while the dynamic RAM receives electrical power and is operated in accordance with its design, for example during a period of time during which the computer system 380 is turned on and operational, the dynamic RAM stores information that is written to it. Similarly, the processor 382 may comprise an internal RAM, an internal ROM, a cache memory, and/or other internal non-transitory storage blocks, sections, or components that may be referred to in some contexts as non-transitory computer readable media or computer readable storage media.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A computer-implemented method of building and training an antibody language model (AbLM) to provide antibody screening or design for virus neutralization, the method comprising:
receiving, by an application stored in a non-transitory memory of a computer system and executed by a processor of the computer system, unlabeled data comprising single-chain protein sequences;
training, by the application, a protein language model (pLM) using the unlabeled data;
initializing, by the application, an AbLM using the trained pLM;
applying, by the application, complementary-determining region (CDR) masking to a plurality of variable heavy (VH) and variable light (VL) chain sequences;
training, by the application, the AbLM using the plurality of CDR-masked VH and VL chain sequences in paired form; and
applying, by the application, the trained AbLM for at least one of antibody screening or antibody design.

2. The method of claim 1, wherein the training the AbLM comprises:
processing an individual pair of the CDR-masked VH and VL chain sequences separately using respective ones of two identical pLMs to generate corresponding VH embeddings and VL embeddings; and
applying a cross-attention fusion model to the VH embeddings and the VL embeddings.

3. The method claim 1, wherein the applying the CDR masking comprises randomly selecting one CDR region and masking all amino acids within the one CDR region.

4. The method of claim 1, wherein the training the AbLM further comprises:
processing a pair of the CDR-masked VH and VL chain sequences using the AbLM to generate an output; and
adapting one or more parameters of the AbLM based on an error measurement between the output of the AbLM and the pair of the CDR-masked VH and VL chain sequences.

5. The method of claim 1, wherein the applying the trained AbLM for the at least one of the antibody screening or the antibody design comprises:
providing an antibody sequence comprising a VH chain sequence and a VL chain sequence as an input to the trained AbLM; and
receiving, from the trained AbLM, an output comprising at least one of embeddings representative of the antibody sequence or a probability distribution of amino acids at each sequence position of the antibody sequence.

6. The method of claim 5, wherein the applying the trained AbLM for the at least one of the antibody screening or the antibody design further comprises:
predicting, by the application, at least one activity for the antibody sequence based on the output of the trained AbLM.

7. The method of claim 6, wherein the predicting the at least one activity for the antibody sequence is based on an activity prediction model trained using labeled data comprising antibody sequences and corresponding label information indicative of response activities associated with a target virus.

8. The method of claim 5, wherein the applying the trained AbLM for the at least one of the antibody screening or the antibody design further comprises:
generating, by the application, a second antibody sequence based on sampling one or more probability distributions of amino acids at one or more sequence positions of the antibody sequence.

9. The method of claim 5, further comprising:
retraining, by the application, the AbLM using at least one of experimental feedback from experiments or reinforcement learning based on the output from the trained AbLM.

10. A computer-implemented method of performing machine learning-based antibody selection for a target virus using an antibody language model (AbLM), the method comprising:
receiving, by an application stored in a non-transitory memory of a computer system and executed by a processor of the computer system, a plurality of antibody sequences;
encoding, by the application, the plurality of antibody sequences into respective feature representations using the AbLM;
for each of the plurality of antibody sequences, predicting, by the application, based on the encoding, respective activity against a target virus using an activity prediction model; and
selecting at least one antibody sequence of the plurality of antibody sequences based on respective predicted activity against the target virus.

11. The method of claim 10, wherein the selected at least one antibody sequence is experimentally tested against the target virus.

12. The method of claim 10, further comprising:
adapting, by the application, one or more parameters of the AbLM based on results of experimentally testing the selected at least one antibody sequence against the target virus.

13. The method of claim 10, wherein the selected at least one antibody sequence is present in a composition further comprising a pharmaceutically acceptable carrier or excipient.

14. The method of claim 10, wherein:
the AbLM is trained using unlabeled data comprising at least one of protein sequences or antibody sequences and the activity prediction model is trained using first labeled data comprising first antibody sequences and respective first activity information associated with a target virus, or
the AbLM and the activity prediction model are jointly trained using second labeled data comprising second antibody sequences and respective second activity information associated with a target virus.

15. A computer-implemented method of using an antibody language model (AbLM) for antibody design, the method comprising:
receiving, by an application stored in a non-transitory memory of a computer system and executed by a processor of the computer system, an antibody sequence;
masking, by the application, a portion of the antibody sequence;
initiating, by the application, an AbLM to process the antibody sequence;
receiving, by the application, from the AbLM, one or more outputs comprising at least one of encoded features of the antibody sequence or a probability distribution of amino acid at each sequence position of the antibody sequence; and
generating, by the application, one or more antibodies for a target virus based on the one or more outputs of the AbLM.

16. The method of claim 15, wherein the masking the portion of the antibody sequence is based on complementary-determining region (CDR) masking.

17. The method of claim 15, wherein the antibody sequence comprises an experimentally generated antibody sequence.

18. The method of claim 15, wherein the generating the one or more antibodies for the target virus is further based on sampling one or more probability distributions of amino acids at one or more sequence positions within the masked portion of the antibody sequence.

19. The method of claim 15, further comprising:
adapting, by the application, one or more parameters of the AbLM based on experimentally testing the one or more generated antibodies against the target virus.

20. The method of claim 15, wherein at least one of the one or more generated antibodies is present in a composition further comprising a pharmaceutically acceptable carrier or excipient.
